(19) United States
(12) Patent Application Publication
(10) Pub. No.: US 2025/0002974 A1
(43) Pub. Date: Jan. 2, 2025

(54) PROTECTING FULLY FUNCTIONAL NUCLEOTIDES FROM DEGRADATION

(71) Applicant: Illumina, Inc., San Diego, CA (US)

(72) Inventors: Sherman Lauw, Singapore (SG); Trina Osothprarop, San Diego, CA (US); Henry Day, Newmarket (GB); Johan Basuki, Singapore (SG); Claire Bevis-Mott, Cambridge (GB); Hester Beard, Cambridge (GB); Chol Steven Yun, San Diego, CA (US); Faye Huber, San Diego, CA (US); Patrizia Iavicoli, Poway, CA (US); Yin Nah Teo, Singapore (SG); Mei Xian Agnes Lee, Singapore (SG); Denise Ng, Singapore (SG)

(73) Assignee: Illumina, Inc., San Diego, CA (US)

(21) Appl. No.: 18/758,066

(22) Filed: Jun. 28, 2024

Related U.S. Application Data
(60) Provisional application No. 63/511,391, filed on Jun. 30, 2023.

(51) Int. Cl. C12Q 1/6806; C12Q 1/6874

(52) U.S. Cl. CPC C12Q 1/6806; C12Q 1/6874

(57) ABSTRACT

This application relates to methods and compositions for protecting fully functional nucleotides (ffNs) against degradation. In some examples, micelles or surfactants are used to protect ffNs. In some examples, bulky cation compounds are used to protect ffNs. In some examples, the compositions that protect ffNs are included in lyophilized material.

### CROSS-REFERENCE TO RELATED APPLICATIONS

This application claims the benefit of U.S. Provisional Patent Application No. 63/511,391, filed on Jun. 30, 2023 and entitled “Protecting Fully Functional Nucleotides from Degradation,” the entire contents of which are incorporated by reference herein.

### FIELD

This application relates to methods and compositions for protecting fully functional nucleotides against degradation.

### BACKGROUND

Many sequencing reagents, in particular fully functional nucleotides (ffNs), suffer from poor stability and therefore need to be handled and stored at lowered temperatures. This incurs additional energy, equipment and material costs, and/or time due to the need for refrigeration, dry ice and/or insulation packaging. One method to improve stability is through lyophilization. However, the improved stability that is obtained through lyophilization is dependent on the lyophilized material being maintained in a dry state.

### SUMMARY

Some examples herein provide a method of stabilizing fully functional nucleotides (ffNs), the method including a step of including the ffNs and at least one surfactant into lyophilized material.

In some examples, the concentration of the ffNs is between about 10 μM and about 15,000 μM. In some examples, the concentration of the at least one surfactant is between about 0.5% and about 2.5%.

In some examples, including the ffNs and the at least one surfactant into the lyophilized material includes freeze-drying the ffNs. In some examples, the ffNs are encapsulated in a hydrophobic core of the surfactant, which inhibits or prevents hydrolysis of the ffNs.

In some examples, the at least one surfactant includes any one or more of tricosaethylene glycol dodecyl ether, poloxamer 407, CHAPS, polyoxyethylene sorbitan monolaurate, and polyoxyethylene sorbitan monopalmitate.

Some examples herein provide a method of stabilizing a fully functional nucleotides (ffNs), the method including a step of including the ffNs and at least one bulky cation compound into lyophilized material.

In some examples, the concentration of the ffNs is between about 10 μM and about 15,000 μM. In some examples, the concentration of the at least one bulky cation compound is between about 1 mM and about 40 mM. In other examples, the concentration of the at least one bulky cation compound is between about 40 mM and about 200 mM. Illustratively, the concentration of the at least one bulky cation compound may be between about 90 mM and about 200 mM.

In some examples, including the ffNs and the at least one bulky cation compound into the lyophilized material includes freeze-drying the ffNs.

In some examples, an ionic interaction is produced between a positive charge on the bulky cation compound and a negative charge on one or more phosphate groups of the ffNs. In some examples, the ionic interaction stabilizes the ffNs through steric hindrance, which inhibits or prevents hydrolysis of the ffNs. In some examples, the ionic interaction stabilizes the ffNs through charge neutralization that reduces the ffNs electrophilicity to water, which inhibits or prevents hydrolysis of the ffNs.

In some examples, the at least one bulky cation compound comprises any one or more of an imidazolium compound, a pyridinium compound, and a quaternary ammonium compound. In some examples, the imidazolium compound includes 1-ethyl-2-methylimidazolium chloride (EMICL), 1-butyl-3-methylimidazolium chloride (BUMICL), 1-butyl-3-methylimidazolium bromide (BUMIBR), or 1-benzyl-3-methylimidazolium chloride (BZMICL). In some examples, the pyridinium compound includes 1-butylpyridinium bromide (BPB), 2-chloro-1-methlypyridinium iodide (CMPI), or 1-ethyl-4-(methoxycarbonyl)pyridinium iodide (EMPI). In some examples, the quaternary ammonium compound includes ammonium chloride (ACL), choline chloride (CCL), tetrabutylammonium chloride (TBACL), tetramethylammonium chloride (TMACL), bis(2-hydroxyethyl)dimethylammonium chloride (DMACL), tris(2-hydroxyethyl)methylammonium methylsulfate (THEMAMS) tributylmethylammonium chloride (TBMACL), trimethylphenylammonium chloride (TMPACL), or diallyldimethylammonium chloride (DADMAC). In one nonlimiting example, the bulky cation compound is or includes TBMACL.

Some examples herein provide a method of preparing ffNs for sequencing, the method including mixing the ffNs with at least one surfactant in a solution; diluting a concentration of the ffNs in a sequencing mix relative to a concentration of the ffNs in the solution by a factor of between about 10× and about 200×; and diluting the concentration of the at least one surfactant in the sequencing mix relative to the concentration of the at least one surfactant in the solution, by a factor of between about 10× and about 1,000×.

In some examples, the concentration of the ffNs in the solution is between about 10 μM and about 15,000 μM, and the concentration of the ffNs in the sequencing mix is between about 5 μM and about 9 μM. In some examples, the concentration of the at least one surfactant in the solution is between about 1.5% and about 2.5%, and the concentration of the at least one surfactant in the sequencing mix is between about 0.05% and about 0.25%.

In some examples, the at least one surfactant includes any one or more of tricosaethylene glycol dodecyl ether, poloxamer 407, CHAPS, polyoxyethylene sorbitan monolaurate, and polyoxyethylene sorbitan monopalmitate.

In some examples, the method further includes performing a sequencing reaction through incorporating the ffNs onto a nucleic acid. In some examples, the sequencing reaction includes a sequencing by synthesis reaction.

Some examples herein provide a method of preparing ffNs for sequencing, including mixing the ffNs with at least one bulky cation compound in a solution; diluting a concentration of the ffNs in a sequencing mix relative to a concentration of the ffNs in the solution by a factor of between about 10× and about 200×; and diluting the concentration the at least one bulky cation compound in the sequencing mix relative to the concentration of the at least one bulky cation compound in the solution, by a factor of between about 100× and about 1,000×.

In some examples, the concentration of the ffNs in the solution is between about 10 μM and about 15,000 μM, and the concentration of the ffNs in the sequencing mix is between about 5 μM and about 9 μM. In some examples, the concentration of the at least one bulky cation compound in the solution is between about 1 mM and about 40 mM, and the concentration of the at least one bulky cation compound in the sequencing mix is between about 0.005 mM and about 4.0 mM. In other examples, the concentration of the at least one bulky cation compound in the solution is between about 40 mM and about 200 mM, e.g., between about 90 mM and about 200 mM. In other examples, the concentration of the at least one bulky cation compound in the sequencing mix is between about 40 mM and about 200 mM, e.g., between about 90 mM and about 200 mM.

In some examples, the at least one bulky cation compound includes any one or more of an imidazolium compound, a pyridinium compound, and a quaternary ammonium compound.

In some examples, the method further includes performing a sequencing reaction through incorporating the ffNs onto a nucleic acid. In some examples, the sequencing reaction comprises a sequencing by synthesis reaction.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF THE DRAWINGS

FIG. 1 illustrates an example a fully function nucleotide (ffN) that includes the functional groups of the triphosphate, the fluorophore, and the blocks.

FIG. 2 schematically illustrates an example of a micelle in an aqueous media.

FIG. 3 schematically illustrates an example of encapsulation of an ffN within a hydrophobic core of a micelle.

FIG. 4 schematically illustrates examples of surfactants that can be used to improve the stability of ffNs.

FIG. 5 provides data showing the percent diphosphate formation within the ffNs after stress, when the ffNs were staged in the presence of various surfactants.

FIG. 6 provides data showing the percent of ffNs that remained intact after stress, when the ffNs were staged in the presence of various surfactants.

FIG. 7 schematically illustrates an example of triphosphate stabilization with bulky cations.

FIG. 8 provides examples of chemical structures of imidazolium compounds.

FIG. 9 provides examples of chemical structures of pyridinium compounds.

FIGS. 10A and 10B provide examples of chemical structures of quaternary ammonium compounds.

FIG. 11 provides data showing the percent diphosphate ffC detected via HPLC after staging for 45° C. for 7 days (7D) in the presence of tetrabutylammonium chloride (TBACL) (Solution format; n=2; Ctrl=Control sample with no TBACL).

FIG. 12 provides data showing the percent diphosphate ffC detected via HLPC after staging for 60° C. for 3 days (3D) in the presence of 30 mM imidazolium additive (lyophilized cakes; n=2; CTRL=control sample with no additive).

FIG. 13 provides data showing the percent diphosphate ffC detected via HLPC after staging for 60° C. for 3 days (3D) in the presence of 30 mM pyridinium additives (lyophilized cakes; n=2; CTRL=control with no additive).

FIG. 14 provides data showing the percent diphosphate ffC detected via HLPC after staging for 60° C. for 3 days (3D) in the presence of 30 mM quaternary additives (lyophilized cakes; n=2; CTRL=control with no additive).

FIG. 15 schematically illustrates an example workflow in which spray-freeze-dried microspheres are resuspended into solution followed by dilution into an incorporation mix.

FIG. 16 provides data showing a FRET polymerase incorporation assay in which 0.2% of various surfactants were used (assuming 10× dilution from microspheres into IMX).

FIG. 17 provides data showing a FRET polymerase incorporation assay with 3 mM of imidazolium and pyridinium cations (assuming 10× dilution from microspheres into IMX).

FIG. 18 provides data showing a FRET polymerase incorporation assay with 3 mM of quaternary ammonium cations (assuming 10× dilution from microspheres into IMX).

FIG. 19 provides an example of a bulky cation-diallydimethylammonium chloride (DADMAC).

FIGS. 20A and 20B provide data of chromatograms showing effect of DADMAC on the purity of various ffNs, under high temperatures.

FIG. 21 provides data showing that, in the presence of heat stress, DADMAC increases the stability of the ffA and reduces diphosphate and monophosphate formation.

FIG. 22 provides data showing that bulky cation compounds provide greater protection for triphosphates in ffNs, as heat stress increases.

FIG. 23 provides data showing that as heat stress increases, micelles impact on the stability of ffNs increases.

FIG. 24 provides data showing that addition of the cationic compound Tributylmethylammonium chloride (TBMACL) to ffNs in a dry state increases stability of triphosphate on the ffNs.

FIG. 25 provides data showing that the addition of CHAPS as well as CHAPS and TBMACL to ffNs in a liquid state increases the stability of intact ffNs.

FIGS. 26-27 provide data showing use of TBMACL to inhibit bacterial growth.

### DETAILED DESCRIPTION

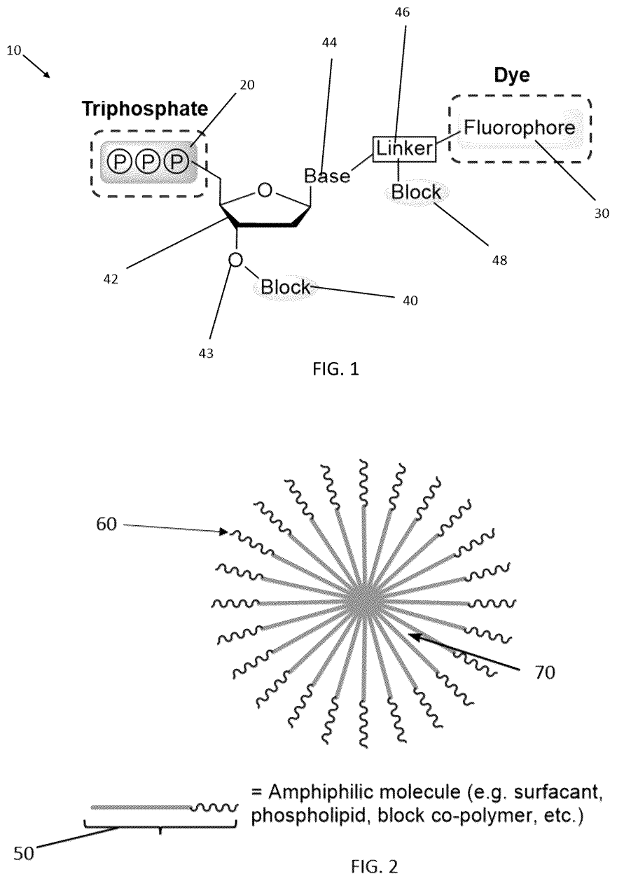

In examples such as illustrated in FIG. 1, a fully functional nucleotide (ffN) (10) is a nucleotide that contains a polyphosphate group (20) (e.g., triphosphate group) that is on the 5′ end of the ffN. The ffN (10) can include a sugar (42) and a 3′-O (43) on the sugar. In some examples, there is a block (40) on the 3′-O (43) of the sugar (42). In the illustrated example, the ffN (10) includes a nucleobase (44) that is connected to a label (e.g., a fluorophore (dye)) (30), through a linker (46). In some examples, the linker (46) is cleavable. In some examples, the linker (46) is cleaved after the ffN (10) is incorporated into a nucleotide strand during a sequencing reaction. Optionally, the ffN (10) can include more than one blocking group. In some examples, a block (48) is on the linker 46. Alternatively, a block can be on the nucleobase (44). In some examples, a block is on any one or more of the linker (46), the 3′—O group (43) and the nucleobase (44).

ffNs can suffer from instability. In particular, ffNs are susceptible to hydrolysis of one or more of the phosphate groups within the polyphosphate group (20), for example under thermal stress. An example degradation mechanism is for a triphosphate moiety to be hydrolyzed to diphosphate and monophosphate. This degradation reduces the effective concentration of ffNs and has a negative impact on sequencing, which can be observed through an increase in phasing and error rate. Additionally, or alternatively, ffNs can be destabilized through hydrolysis of their labels (30) (e.g., fluorophores). Hydrolysis can result from water/moisture in the environment.

Additionally, where reagent ambient stability requirements increase, the risk of bioburden grown in liquid reagents also increases. This risk has previously been mitigated through the addition of biocides such as methyisothiazolinone (MIT) and ProClin™ (preservative commercially available from Millipore Sigma). However, these biocides are costly and have may negatively impact sequencing by synthesis.

Lyophilization is a previously known method to stabilize ffNs. However, even trace amounts of water in contact with ffNs following lyophilization can cause hydrolysis. Thus, in order for lyophilization to fully maintain ffN stability, the materials preferably remain in a completely dry state until they are ready for use. Disclosed herein are reagents that can be used to improve the stability of ffNs. In some examples, micelles are used to increase ffN stability. In some examples, bulky cation compounds are used to increase ffN stability. In some examples, the bulky cation compound TBMACL may also be used as a biocide to reduce or eliminate the need for inclusion of a separate biocidal compound.

First, some terms used herein will be briefly explained. Then, some example compositions and example methods will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10% of the stated value, such as less than or equal to ±5% of the stated value, such as less than or equal to ±2% of the stated value, such as less than or equal to ±1% of the stated value, such as less than or equal to ±0.5% of the stated value, such as less than or equal to ±0.2% of the stated value, such as less than or equal to ±0.1% of the stated value, such as less than or equal to ±0.05% of the stated value.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 5-halouracil, 5-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the phrase “fully functional nucleotide (ffN)” refers to a nucleotide that contains a polyphosphate group (e.g., a triphosphate group) and nucleobase that is coupled to a label, e.g., is linked to a fluorophore using a linker. In some examples, the linker is cleavable. In some examples, the ffN contains a sugar with a 3′—O group. In some examples, the ffN contains at least one block. In some examples, there is a block on any one or more of the linker, the 3′—O group, and the nucleobase. A ffN can include any nucleobase. The terms “ffA,” “ffC,” “ffIT,” and “ffG” refer to ffNs that include the adenosine, cytosine, thymine, and guanine nucleobases, respectively.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “micelle” refers to an aggregate of molecules that are suspended in a fluid. A micelle is typically between approximately 10 nm and 100 nm in size. A micelle is amphiphilic containing hydrophobic and hydrophilic regions.

As used herein, the term “surfactant” refers to molecules that are amphiphilic. An aggregate of surfactants dispersed in a fluid produces a micelle.

As used herein, the phrase “bulky cation compound” refers to a positively charged compound that contains more than two atoms or at least one functional group. The phrases “bulky cation compound” and “bulky cation” have the same meaning and are used interchangeably herein. Examples of “bulky cation compounds” include pyridinium compounds, imidazolium compounds, and quaternary ammonium compounds.

As used herein, the term “microsphere” refers to a particle between about 1 μM and 1,000 μM. In some examples, the particle is lyophilized.

As used herein, the term “amphiphilic” is intended to mean having both hydrophilic and hydrophobic properties.

As used herein, the term “lyophilized material” refers to any material that has been freeze dried. In some examples, the material includes a ffN and a bulky cation compound. In some examples, the material includes a ffN and a micelle. In some examples, the material includes a ffN and a surfactant. In some examples, the material includes a ffN, a bulky cation compound, and a surfactant.

Methods of Stabilizing ffNs using Bulky Cation Compounds

Some examples herein provide a method of stabilizing ffNs using bulky cation compounds. In some examples, an ionic interaction is produced between the positive charge on the bulky cation compound and the negative charge on one or more of the phosphates of the ffNs.

In some examples, the ionic interaction produces stabilized ffNs through steric hindrance that inhibits or prevents water from accessing and hydrolyzing the ffNs. In some examples, this ionic interaction produces stabilized ffNs through charge neutralization. For example, the ionic interaction reduces the electrophilicity of the phosphates to nucleophiles such as water, which inhibits or prevents water from accessing and hydrolyzing the ffNs.

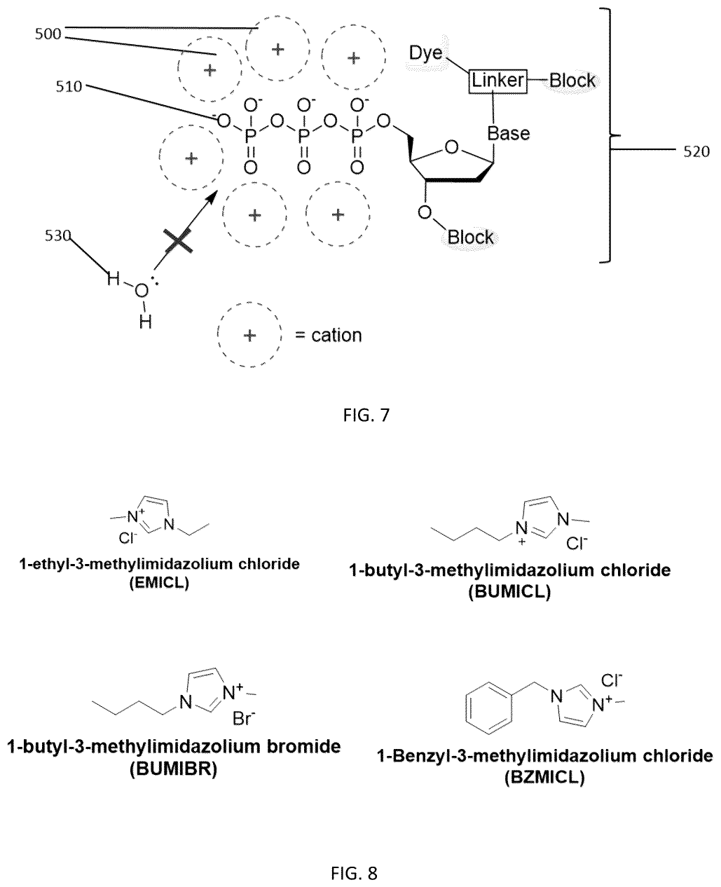

An example schematic of an interaction between a bulky cation compound and a ffN is shown in FIG. 7. Bulky cation compounds 500 ionically interact with phosphate groups 510 on ffNs 520. This ionic interaction between the bulky cation compounds 500 and the phosphate groups 510 prevents or inhibits water 530 from interacting with the ffN 520. Because water 530 does not interact with the ffN 520, the phosphate groups 510 are not hydrolyzed, and therefore are stabilized.

In some examples, bulky cation compounds and ffNs are lyophilized to produce lyophilized material. In some examples, the bulky cation compounds and ffNs are lyophilized into microspheres or lyophilized cakes. In some examples, the bulky cation compounds and ffNs are lyophilized through spray-freeze-drying. In some examples, the bulky cation compounds and ffNs are lyophilized through any lyophilization technique known in the art.

In some examples, the concentration of the ffNs after spray-freeze-drying is between about 70 μM and about 1,400 μM, for example, between about 70 μM and about 100 μM, between about 100 μM and about 200 μM, between about 200 μM and about 300 μM, between about 300 μM and about 400 μM, between about 400 μM and about 500 μM, between about 500 μM and about 600 μM, between about 600 μM and about 700 μM, between about 700 μM and about 800 μM, between about 800 μM and about 900 μM, between about 900 μM and about 1,000 μM, between about 1,000 μM and about 1,100 μM, between about 1,100 μM and about 1,200 μM, between about 1,200 μM and about 1,300 μM, between about 1,300 μM and about 1,400 μM.

In some examples, the concentrations of the ffNs after spray-freeze-drying is between about 10 μM and about 15,000 μM, for example, between about 10 μM and 100 μM, between about 100 μM and 1,000 μM, between about 1,000 μM and about 2,000 μM, between about 2,000 μM and about 3,000 μM, between about 3,000 μM and about 4,000 μM, between about 4,000 μM and about 5,000 μM, between about 5,000 μM and about 6,000 μM, between about 7,000 μM and about 8,000 μM, between about 8,000 μM and about 9,000 μM, between about 9,000 μM and about 10,000 μM, between about 10,000 μM and about 11,000 μM, between about 11,000 μM and about 12,000 μM, between about 12,000 μM and about 13,000 μM, between about 13,000 μM and about 14,000 μM, or between about 14,000 μM and about 15,000 μM.

In some examples, the concentration of the bulky cation compounds after spray-freeze-drying is optimized to protect the ffNs from degradation. In some examples, the concentration of the bulky cation compounds after spray-freeze-drying is between about 1 mM and about 40 mM, for example, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 33 mM, about 34 mM, about 35 mM, about 36 mM, about 37 mM, about 38 mM, about 39 mM, or about 40 mM. In other examples, the concentration of the bulky cation compounds after spray-freeze-drying is between about 40 mM and about 200 mM, e.g., between about 90 mM and about 200 mM.

In some examples, after spray-freeze-drying the ffNs and the bulky cation compounds into the microspheres, the microspheres are stored. In some examples, the microspheres are stored for 6 months or less. In some examples, the microspheres are stored for about 6 months or less at a temperature of between about 25° C. and about 35° C., for example, about 25° C., about 26° C., about 27° C., about 28° C., about 29° C., about 30° C., about 31° C., about 32° C., about 33° C., about 34° C., or about 35° C. In some examples, the microspheres are stored for about 6 months or more, for example, at least about 6 months, at least about 1 year, at least about 2 years, at least about 3 years, at least about 4 years, at least about 5 years, at least about 6 years, at least about 7 years, at least about 8 years, at least about 9 years, or at least about 10 years. In some examples, the microspheres are stored for longer than about 10 years. In some examples, the microspheres are stored for about 6 months or more at a temperature of between about 5° C. and about 10° C., for example, about 5° C., about 6° C., about 7° C., about 8° C., about 9° C., or about 10° C.

Methods of Sequencing Using Bulky Cation Compounds and ffNs

In some examples, after spray-freeze-drying of the ffNs and bulky cation compounds into microspheres, a sequencing reaction is performed. In some examples, the ffNs and bulky cation compounds are resuspended into a sequencing mix, prior to the sequencing reaction. For details regarding the components of example sequencing mixes into which ffNs can be resuspended see: U.S. Patent Application Publication No. 2022/0220553, filed on Mar. 30, 2022 and entitled “Nucleotides with a 3′ AOM Blocking Group”; U.S. Patent Application Publication No. 2021/0403500, filed on Jun. 21, 2021 and entitled “Nucleosides and nucleotides with 3′ acetal blocking group”; and U.S. Patent Application Publication No. 2022/0396832, filed May 19, 2022 and entitled “Compositions and methods for sequencing by synthesis,” the entire contents of each of which are incorporated by reference herein.

In some examples, resuspension of the ffNs and the bulky cations into the sequencing mix causes (i) a reduction in concentration of the ffNs, (ii) a reduction in concentration of the bulky cation compounds, or (iii) a reduction in both the ffNs and the bulky cation compounds.

In some examples, the concentration of the ffNs in the sequencing mix is between about 5.0 μM and about 9.0 μM, for example, about 5.0 μM, about 5.1 μM, about 5.2 μM, about 5.3 μM, about 5.4 μM, about 5.5 μM, about 5.6 μM, about 5.7 μM, about 5.8 μM, about 5.9 μM, about 6.0 μM, about 6.1 μM, about 6.2 μM, about 6.3 μM, about 6.4 μM, about 6.5 μM, about 6.6 μM, about 6.7 μM, about 6.8 μM, about 6.9 μM, about 7.0 μM, about 7.1 μM, about 7.2 μM, about 7.3 μM, about 7.4 μM, about 7.5 μM, about 7.6 μM, about 7.7 μM, about 7.8 μM, about 7.9 μM, about 8.0 μM, about 8.1 μM, about 8.2 μM, about 8.3 μM, about 8.4 μM, about 8.5 μM, about 8.6 μM, about 8.7 μM, about 8.8 μM, about 8.9 μM, or about 9.0 μM.

In some examples, the concentration of the bulky cation compounds in the sequencing mix is between about 0.0025 mM and about 4.5 mM, for example, between about 0.0025 mM and about 0.0050 mM, between about 0.0050 mM and about 0.0075 mM, between about 0.0075 and about 0.01, between about 0.01 mM and about 0.02 mM, between about 0.02 mM and about 0.03 mM, between about 0.03 mM and about 0.04 mM, between about 0.04 mM and about 0.05 mM, between about 0.05 mM and about 0.06 mM, between about 0.06 mM and about 0.07 mM, between about 0.07 mM and about 0.08 mM, between about 0.08 mM and about 0.09 mM, between about 0.09 mM and about 0.1 mM, between about 0.1 mM and about 0.2 mM, between about 0.2 mM and about 0.3 mM, between about 0.3 mM and about 0.4 mM, between about 0.4 mM and about 0.5 mM, between about 0.5 mM and about 0.6 mM, between about 0.6 mM and about 0.7 mM, between about 0.7 mM and about 0.8 mM, between about 0.8 mM and about 0.9 mM, between about 0.9 mM and about 1.0 mM, between about 1.0 mM and about 1.5 mM, between about 1.5 mM and about 2.0 mM, between about 2.0 mM and about 2.5 mM, between about 2.5 mM and about 3.0 mM, between about 3.0 mM and about 3.5 mM, between about 3.5 mM and about 4.0 mM, or between about 4.0 mM and about 4.5 mM. In other examples, the concentration of the bulky cation compounds in the sequencing mix is between about 40 mM mM and about 200 mM, e.g., between about 90 mM and about 200 mM.

In some examples, the ffNs are diluted in a sequencing mix relative to the concentration of the ffNs in the solution, by a factor of between about 10-fold (10×) and 200-fold (200×), for example, between about 10-fold and about 50-fold, between about 50-fold and about 100-fold, between about 100-fold and about 150-fold, or between about 150-fold and about 200-fold.

In some examples, the at least one bulky cation compound is diluted in the sequencing mix relative to the concentration of the at least one bulky cation compound in the solution by a factor of between about 100-fold (100×) and about 1,000-fold (1000×), for example between about 100-fold and about 200-fold, between about 200-fold and about 300-fold, between about 300-fold and about 400-fold, between about 400-fold and about 500-fold, between about 500-fold and about 600-fold, between about 600-fold and about 700-fold, between about 700-fold and about 800-fold, between about 800-fold and about 900-fold, or between about 900-fold and about 1,000-fold.

In some examples, diluting the at least one bulky cation compound in the sequencing mix causes the at least one bulky cation compound to dissociate from the ffNs, which allows the ffNs to be released. In some examples, the released ffNs can be used as substrates in sequencing reactions such as, for example, sequencing by synthesis reactions.

Methods of Stabilizing ffNs Using Micelles

Some examples herein provide methods of stabilizing ffNs using micelles. In some examples, the micelles stabilize the ffNs in aqueous media. The micelle is amphiphilic and therefore has both hydrophobic and hydrophilic properties. An example of a micelle is shown in FIG. 2. The amphiphilic molecules 50 of the micelle includes a hydrophilic region 60 and a hydrophobic region 70.

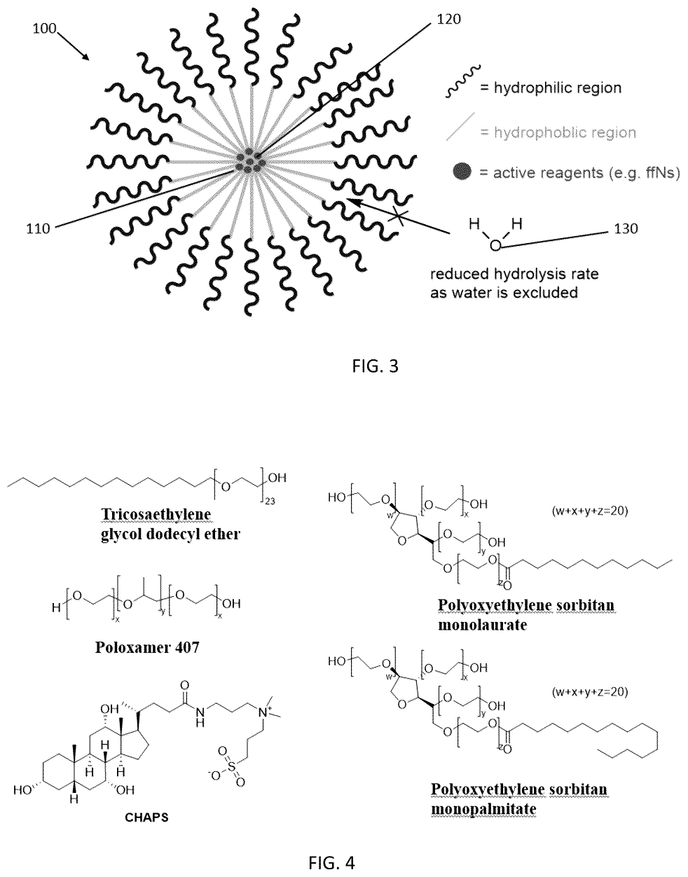

In some examples, the micelles encapsulate the ffNs. In some examples, the micelles encapsulate the ffNs in the hydrophobic core of the micelles. FIG. 3 provides an example schematic of a mechanism of micelles encapsulating ffNs. The micelle 100 contains a hydrophobic core 110 where the active reagents such as ffNs 120 are encapsulated. Because water 130 does not interact with the hydrophobic core 110 of the micelle 100, the ffNs 120 are not hydrolyzed.

In some examples, micelle(s) that have encapsulated ffNs are lyophilized to produce lyophilized material. In some examples, the micelle(s) and ffNs are lyophilized into microspheres or lyophilized cakes. In some examples, the micelle(s) and ffNs are lyophilized through spray-freeze-drying. In some examples, the micelle(s) and ffNs are lyophilized through any lyophilization technique known in the art.

In some examples, spray-freeze-drying the micelle or micelles into microspheres produces a concentration of ffNs that is between about 70 μM and about 1,400 μM, for example, between about 70 μM and about 100 μM, between about 100 μM and about 200 μM, between about 200 μM and about 300 μM, between about 300 μM and about 400 μM, between about 400 μM and about 500 μM, between about 500 μM and about 600 μM, between about 600 μM and about 700 μM, between about 700 μM and about 800 μM, between about 800 μM and about 900 μM, between about 900 μM and about 1,000 μM, between about 1,000 μM and about 1,100 μM, between about 1,100 μM and about 1,200 μM, between about 1,200 μM and about 1,300 μM, between about 1,300 μM and about 1,400 μM.

In some examples, the concentrations of the ffNs after spray-freeze-drying is between about 10 μM and about 15,000 μM, for example, between about 10 μM and 100 μM, between about 100 μM and 1,000 μM, between about 1,000 μM and about 2,000 μM, between about 2,000 μM and about 3,000 μM, between about 3,000 μM and about 4,000 μM, between about 4,000 μM and about 5,000 μM, between about 5,000 μM and about 6,000 μM, between about 7,000 μM and about 8,000 μM, between about 8,000 μM and about 9,000 μM, between about 9,000 μM and about 10,000 μM, between about 10,000 μM and about 11,000 μM, between about 11,000 μM and about 12,000 μM, between about 12,000 μM and about 13,000 μM, between about 13,000 μM and about 14,000 μM, or between about 14,000 μM and about 15,000 μM.

In some examples, the concentration of the surfactant or surfactants after spray-freeze-drying is optimized to protect the ffNs from degradation. In some examples, spray-freeze-drying produces a concentration of the surfactant or surfactants between about 0.5% and about 3.0%, for example, between about 0.5% and about 1%, between about 1% and about 1.5%, between about 1.5%, and about 2%, between about 2% and about 2.5%, or between about 2.5%, and about 3%.

In some examples, after spray-freeze-drying of the ffNs and the micelle or micelles into the microspheres, the microspheres are stored.

Methods of Sequencing Using Micelles and ffNs

In some examples, after spray-freeze-drying of the ffNs and micelles into microspheres, a sequencing reaction is performed. In some examples, the ffNs and micelles in the microspheres are resuspended into a sequencing mix, prior to the sequencing reaction.

In some examples, resuspension of the ffNs and the micelles into the sequencing mix causes (i) a reduction in concentration of the ffNs, (ii) a reduction in concentration of the micelles, or (iii) a reduction in both the ffNs and the micelles.

In some examples, the concentration of the ffNs in the sequencing mix is between about 5.0 μM and about 9.0 μM, for example, about 5.0 μM, about 5.1 μM, about 5.2 μM, about 5.3 μM, about 5.4 μM, about 5.5 μM, about 5.6 μM, about 5.7 μM, about 5.8 μM, about 5.9 μM, about 6.0 μM, about 6.1 μM, about 6.2 μM, about 6.3 μM, about 6.4 μM, about 6.5 μM, about 6.6 μM, about 6.7 μM, about 6.8 μM, about 6.9 μM, about 7.0 μM, about 7.1 μM, about 7.2 μM, about 7.3 μM, about 7.4 μM, about 7.5 μM, about 7.6 μM, about 7.7 μM, about 7.8 μM, about 7.9 μM, about 8.0 μM, about, 8.1 μM, about 8.2 μM, about 8.3 μM, about 8.4 μM, about 8.5 μM, about 8.6 μM, about 8.7 μM, about 8.8 μM, about 8.9 μM, or about 9.0 μM.

In some examples, the concentration of the surfactant in the sequencing mix is between about 0.015% and about 2%, for example, between about 0.015% and about 0.1%, between about 0.1% and about 0.2%, between about 0.2% and about 0.3%, between about 0.3% and about 0.4%, between about 0.4% and about 0.5%, between about 0.5% and about 0.6%, between about 0.6% and about 0.7%, between about 0.7% and about 0.8%, between about 0.8% and about 0.9%, between about 0.9% and about 1.0%, between about 1.0% and about 1.1%, between about 1.1% and about 1.2%, between about 1.2% and about 1.3%, between about 1.3% and about 1.4%, between about 1.4% and about 1.5%, between about 1.5% and about 1.6%, between about 1.6% and about 1.7%, between about 1.7% and about 1.8%, between about 1.8% and about 1.9%, or between about 1.9% and about 2.0%.

In some examples, a method of preparing ffNs for sequencing is provided, including mixing ffNs with at least one surfactant in a solution. In some examples, the ffNs are mixed with the at least one surfactant in a microsphere.

In some examples, the ffNs are diluted in a sequencing mix relative to the concentration of the ffNs in the solution, by a factor of between about 10-fold (10×) and 200-fold (200×), for example, between about 10-fold and about 50-fold, between about 50-fold and about 100-fold, between about 100-fold and about 150-fold, between about 150-fold and about 200-fold.

In some examples, the at least one surfactant is diluted in the sequencing mix relative to the concentration of the at least one surfactant in the solution by a factor of between about 10-fold (10×) and about 200-fold (200×), for example between about 10-fold and about 50-fold, between about 50-fold and about 100-fold, between about 100-fold and about 150-fold, between about 150-fold and about 200-fold.

In some examples, diluting the micelles in the sequencing mix causes the concentration of the micelles to drop below a critical micelle concentration (CMC). In some examples, dropping the micelles below CMC allows for release of the ffNs from the micelles such that the ffNs can be used in sequencing reactions. In some examples, the released ffNs are used in sequencing reactions such as, for example, sequencing by synthesis reactions.

### Bulky Cation Compounds and Surfactants

In some examples, the bulky cation compound includes any of a quaternary ammonium compound, an imidazolium compound, a pyridinium compound, a phosphonium compound, a sulfonium compound, or a pyrrolidinium compound.

In some examples, the bulky cation compound includes an imidazolium compound. In some examples, the imidazolium compound includes any one or more of 1-ethyl-3-methylimidazolium chloride (EMICL), 1-butyl-3-methylimidazolium chloride (BUMICL), 1-butyl-3-methylimidazolium bromide (BUMIBR) or 1-benzyl-3-methylimidazolium chloride (BZMICL) (see FIG. 8). In some examples, the imidazolium includes any imidazolium known in the art.

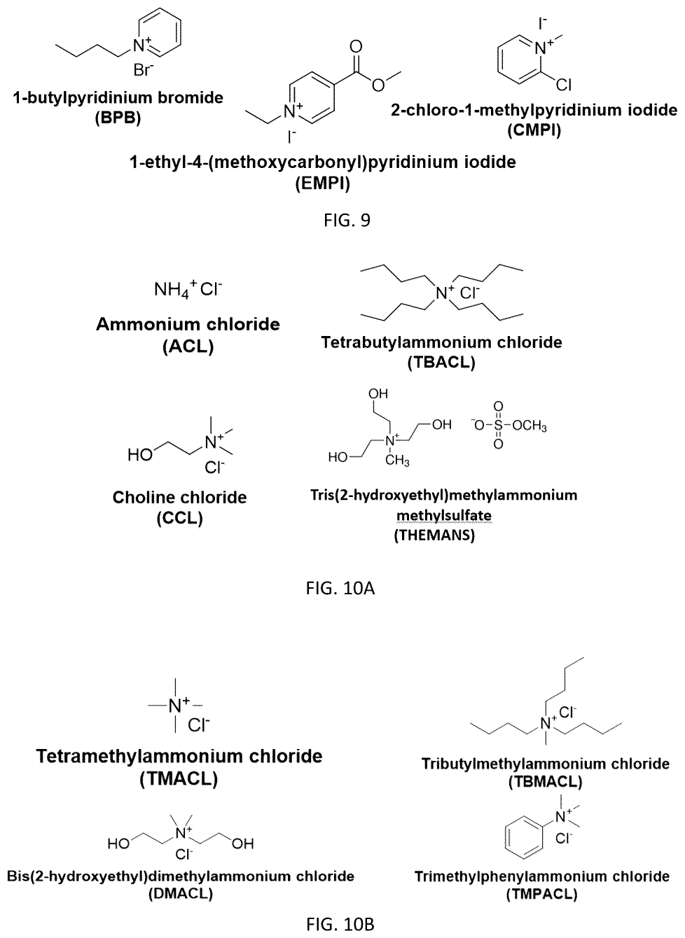

In some examples, the bulky cation compound includes a pyridinium compound. In some examples, the pyridinium compound includes any one or more of 1-butylpyridinium bromide (BPB), 2-chloro-1-methylpyridinium iodide (CMPI), or 1-ethyl-4-(methoxycarbonyl)pyridinium iodide (EMPI) (see FIG. 9).

In some examples, the bulky cation compound includes a quaternary ammonium compound. In some examples, the quaternary ammonium compound includes any one or more of ammonium chloride (ACL), tetramethylammonium chloride (TMACL), tetrabutylammonium chloride (TBACL), tributylmethylammonium chloride (TBMACL), choline chloride (CCL), bis(2-hydroxyethyl)dimethylammonium chloride (DMACL), trimethylphenylammonium chloride (TMPACL) or tris(2-hydroxyethyl)methylammonium methylsulfate (THEMAMS) (see FIGS. 10A and 10B). In some examples, the quaternary ammonium compound includes diallydimethylammonium chloride (DADMAC). In one nonlimiting example, the bulky cation compound is or includes TBMACL.

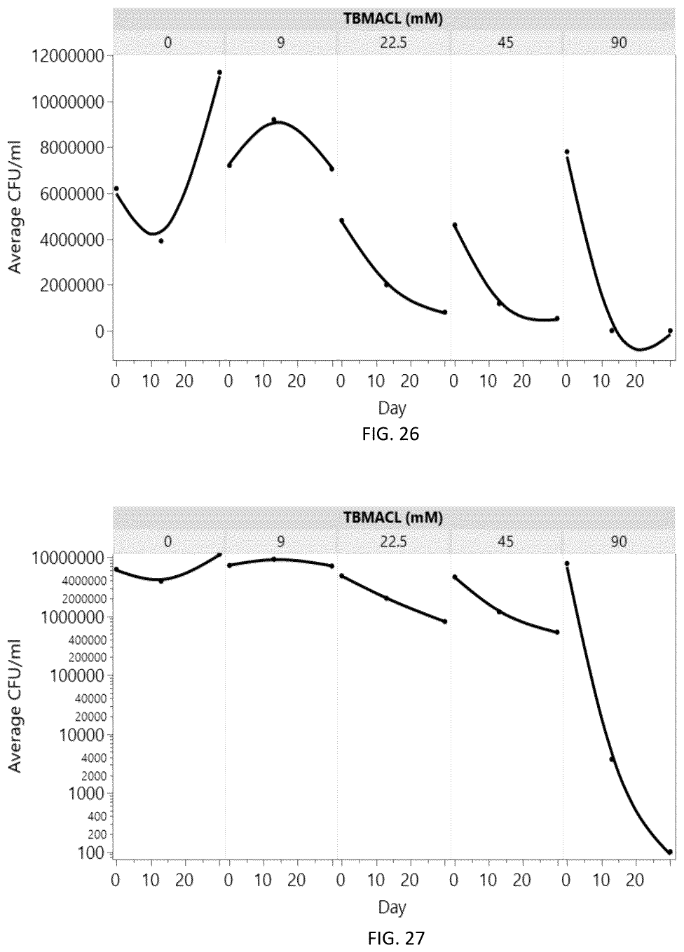

For example, the bulky cation compound TBMACL may help to stabilize ffNs in one or more additional ways. For example, in a manner such as will be described in greater detail below with reference to FIGS. 26 and 27, TBMACL has been demonstrated to possess biocidal properties and may therefore be used to mitigate the risk of bioburden growth within liquid reagents, e.g., without the use of a separate biocide, such as methyisothiazolinone (MIT) and ProClin™ (preservative commercially available from Millipore Sigma), in addition to stabilizing ffNs in a manner which is compatible with sequencing (e.g., sequencing by synthesis).

In some examples, the surfactant includes any one or more of a polysorbate, a sorbitan ester, an alkyl polyglucoside, a poloxamer, an oligo(alkylethylene oxide), or a polysulfobetaine.

In some examples, a surfactant includes tricosaethylene glycol dodecyl ether, poloxamer 407, CHAPS, polyoxyethylene sorbitan monolaurate, and polyoxyethylene sorbitan monopalmitate (see FIG. 4). Poloxamer 407 is a triblock copolymer including a central hydrophobic block of about 56 repeat units of propylene glycol flanked by two hydrophilic blocks each having about 101 repeat units of polyethylene glycol (PEG), although it will be appreciated that poloxamers with other numbers of repeat units of propylene glycol and polyethylene glycol suitably may be used. CHAPS refers to 3-[(3-cholamidopropyl)dimethylammonio]-1-propanesulfonate, and its IUPAC name is 3-{dimethyl[3-(3α,7α,12α-trihydroxy-5β-cholan-24-amido)propyl]azaniumyl}propane-1-sulfonate. The structure for CHAPS is shown in FIG. 4.

In some examples, a surfactant includes one or more of: polyethylene glycol (PEG) stearate, SB-12 (a commercially available zwitterionic, sulfobetaine surfactant), lauramidopropyl betaine, and n-dodecyl-β-D-maltoside. In some examples, the surfactant includes two or more of: polyethylene glycol (PEG) stearate, SB-12, lauramidopropyl betaine, and n-dodecyl-β-D-maltoside. In some examples, the surfactant includes each of: polyethylene glycol (PEG) stearate, SB-12, lauramidopropyl betaine, and n-dodecyl-β-D-maltoside.

### Lyophilization (Freeze Drying)

Lyophilization is a process in which a product is frozen followed by dehydration of the product at low pressure. This process results in transition of the product from a solid phase directly to a gas phase, without passing through a liquid phase. As used herein, methods of lyophilizing include shelf-freeze-drying and spray-freeze-drying to produce lyophilized cakes and/or lyophilized microspheres.

### Spray-Freeze-Drying

Spray-freeze-drying is a process in which a product in a liquid state is converted to a dried powder. Starting with the product in the liquid state, the solution is aerosolized into small droplets and subjected to lowered temperatures to generate frozen beads. These frozen particles are then dehydrated at low pressure to produce the final dried particles.

### Sequencing By Synthesis

The ffNs described herein can be used in sequencing reactions. During a sequencing-by-synthesis reaction, ffNs are incorporated into a polypeptide chain.

Sequencing can be carried out using any suitable “sequencing-by-synthesis” technique, wherein nucleotides (e.g., ffNs) are added successively to the free 3′ hydroxyl group, resulting in synthesis of a polynucleotide chain in the 5′ to 3′ direction. The nature of the nucleotide added is preferably determined after each addition. One particular sequencing method relies on the use of modified nucleotides that can act as reversible chain terminators. Such reversible chain terminators include removable 3′ blocking groups. Once such a modified nucleotide has been incorporated into the growing polynucleotide chain complementary to the region of the template being sequenced there is no free 3′—OH group available to direct further sequence extension and therefore the polymerase cannot add further nucleotides. Once the nature of the base incorporated into the growing chain has been determined, the 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached thereto a different label, known to correspond to the particular base, to facilitate discrimination between the bases added at each incorporation step. Suitable labels are described in PCT application PCT/GB2007/001770, the entire contents of which are incorporated by reference herein. Alternatively, a separate reaction may be carried out containing each of the modified nucleotides added individually.

The modified nucleotides (e.g., ffNs) may carry a label to facilitate their detection. In a particular example, the label is a fluorescent label. Each nucleotide type may carry a different fluorescent label. One method for detecting the fluorescently labelled nucleotides includes using laser light of a wavelength specific for the labelled nucleotides, or the use of other suitable sources of illumination. The fluorescence from the label on an incorporated nucleotide may be detected by a CCD camera or other suitable detection means. Suitable detection means are described in PCT/US2007/007991, the entire contents of which are incorporated by reference herein.

An extension reaction, in which nucleotides are added to the 3′ end of a primer is performed using a polymerase, such as a DNA or RNA polymerase. In one example, the polymerase is a non-thermal isothermal strand displacement polymerase. Suitable non-thermostable strand displacement polymerases according to the present invention can be found, for example, through New England BioLabs, Inc. and include phi29, Bsu, Klenow, DNA Polymerase I (E. coli), and Therminator. A particularly preferred polymerase is Bsu.

### Working Examples

The examples provided below are included for illustrative purposes and are not meant to be limiting of any of the disclosures described herein.

### Example 1-Stabilizing ffNs Using Micelles

In order to demonstrate the feasibility of the abovementioned strategy, a total of 5 different surfactants illustrated in FIG. 4 were examined. Only non-ionic surfactants were tested, due to the potential of ionic surfactants being able to denature proteins such as the polymerases which are vital to incorporating the ffNs onto the growing DNA strands during sequencing. ffN samples were prepared in solution format, and thereafter staged in the presence or absence of the different surfactant additives for a period of 7 days at 45° C. Each of the surfactants was tested at two different concentrations, 0.5% and 2%.

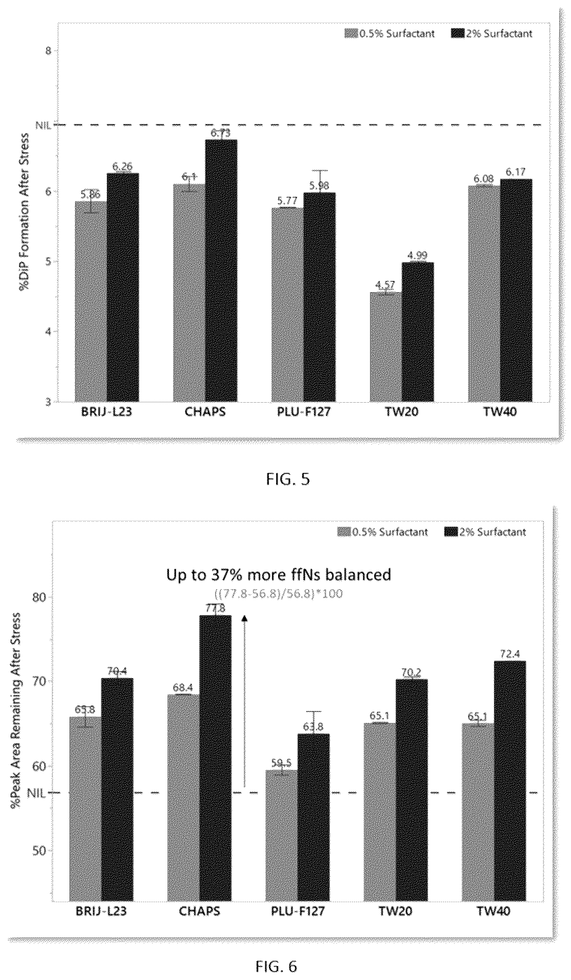

As shown in FIGS. 5 and 6, the presence of the surfactants each displayed a clear benefit towards improving ffN stabilities. For instance, as compared to a nil control (no surfactants added), it can be seen from FIG. 5 that a noticeably lowered degree of dephosphorylation had taken place when the ffNs were staged in the presence of the surfactants tested (i.e. dotted line (control) vs. bars). Moreover, as shown in FIG. 6, when examining the amount of intact/un-degraded ffNs that remained after staging, it is also apparent that a significantly larger percentage of ffNs were able to survive the heat stress as compared to a scenario whereby no surfactants were added. Additionally, a larger concentration of surfactant caused both a lower degree of dephosphorylation (FIG. 5) and an increased amount of intact/un-degraded ffNs (FIG. 6). This was likely due to the larger availability of micelles present in solution that allowed more ffNs to be encapsulated in the micelles.

In FIGS. 5 and 6:
BRIJ-L23=tricosaethylene glycol dodecyl ether
PLU-F127=poloxamer 407
TW20=polyoxyethylene sorbitan monolaurate
TW40=polyoxyethylene sorbitan monopalmitate

Of the surfactants used, CHAPS performed the best in that it was able to increase ffN retention by almost 40% against the control sample (see FIG. 6). Moreover, as the stress condition (45° C. for 7 days) was chosen arbitrarily (sufficiently harsh just so as to ensure reasonable levels of degradation were achieved to enable comparisons), it is expected to be highly likely that improvements in % ffN remaining would increase as staging duration increases.

These results suggest that micellar encapsulation of the ffNs is beneficial in reducing the amount of degradation of both the triphosphate and dye on the ffNs.

### Example 2—Stablizing ffNs Using Bulky Cation Compounds

Various bulky cation compounds were tested to determine whether they stabilized ffNs.

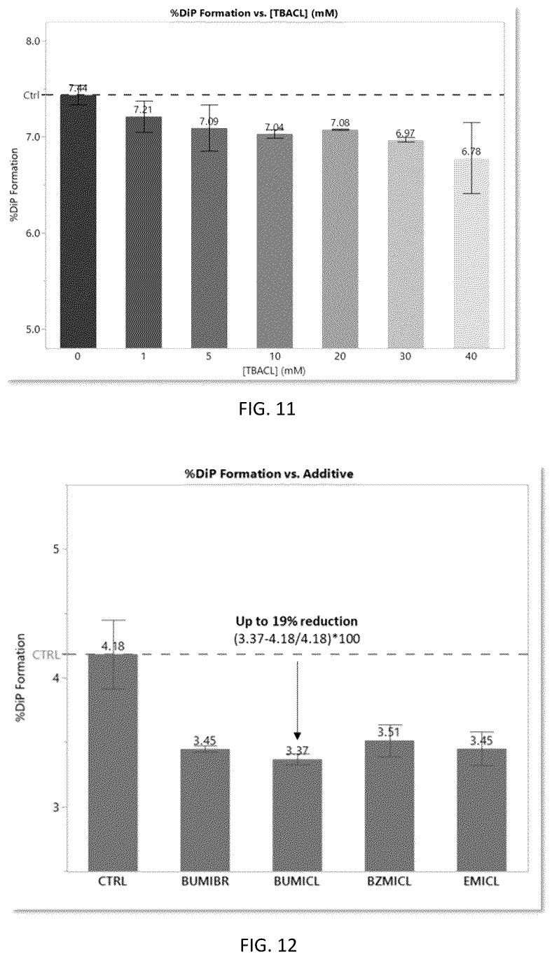

Initially, tetrabutylammonium chloride (TBACL) was selected as a model substrate for use in determining a suitable concentration of additive in subsequent screening experiments. Between 1 to 40 mM of TBACL was tested, and it was found that higher concentrations of TBACL generally also resulted in reduced dephosphorylation (FIG. 11). The concentration of salt present has an inversely proportional relationship to the glass-transition temperatures (Tg/Tg′) and lyophilizability of the reagent. As such, even higher concentrations of cations were not pursued. A concentration of 30 mM cations was used going forward to achieve a balance between both chemical and physical stabilities.

The efficacies of the other cationic substrates were also tested. This was performed using lyophilized ffN cake samples that were formulated with and without said additives, and by staging at an arbitrarily selected stress condition of 60° C. for a period of 3 days.

Overall, as shown by FIGS. 12, 13, and 14, the addition of imidazolium, pyridinium and quaternary ammonium cations generally had a positive effect in increasing triphosphate stability or reducing dephosphorylation. As shown in FIG. 12, when placed in the presence of the imidazolium cations, it was found the amount of diphosphate ffN (a major degradant of triphosphate hydrolysis) formed after heat exposure was reduced by up to 19% as compared to a control sample where no cationic additive was added.

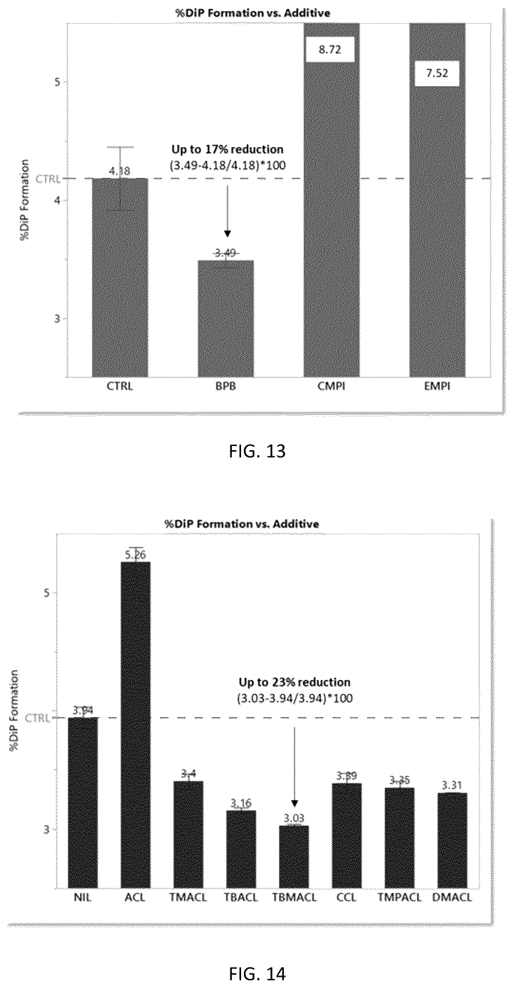

The addition of 1-butylpyridinium bromide (BPB) into the ffN formulation was found to be beneficial in the sense that approximately 17% less dephosphorylation was observed (FIG. 13). 2-chloro-1-methylpyridinium iodide (CMPI) and 1-ethyl-4-(methoxycarbonyl)pyridinium iodide (EMPI), however, appeared to have negatively impacted triphosphate stability (FIG. 13). This is potentially due to CMPI and EMPI being unstable and having undergone degradation themselves which inadvertently also affected the ffNs. These results demonstrate the criticality of cation selection in that it is important to ensure that the additives themselves are stable to heat-stress.

It was also found that the deployment of quaternary ammonium cations was able to induce triphosphate stability (FIG. 14). In particular, of all 14 cationic additives tested, tributylmethylammonium chloride (TBMACL) was found to provide up to a 23% reduction in diphosphate formation of ffNs (FIG. 14). On the other hand, a higher level of diphosphate formation was observed in the presence of ammonium chloride (ACL) likely because this compound had decomposed under the heat stress. Moreover, as the stress condition (60° C. for 3 days) was chosen arbitrarily (sufficiently harsh just to ensure reasonable levels of degradation were achieved to enable comparisons), it is expected to be highly likely that observed improvements in triphosphate stability would increase as staging duration increases too.

DADMAC (diallyldimethylammonium chloride): 100 μM ffNs were stressed in the presence of 10 mM DADMAC (FIG. 19) in 15 mM 3-(N-morpholino)propanesulfonic acid (MOPS) buffer and 5× glycine buffer (250 mM glycine pH 9.9, 250 mM NaCl, 5 mM ethylediaminetetraacetic acid (EDTA)). Each type of ffNs were tested: ffA, ffC, ffT, and ffG.

The samples were staged for 2 days at 40° C. in the glycine buffer, 2 days at 65° C. and 7 days at 4° C. in the MOPS buffer. DADMAC was tested to see if it could be added to the incorporation mix or ffN mixtures without causing any negative interactions with ffNs.

The results showed that DADMAC had no negative effect under the milder stress conditions. However, at high temperatures, stressed FFNs in the presence of DADMAC had a higher % ffN purity as compared to control and lower levels of diphosphate. This was true of all 4 ffNs tested.

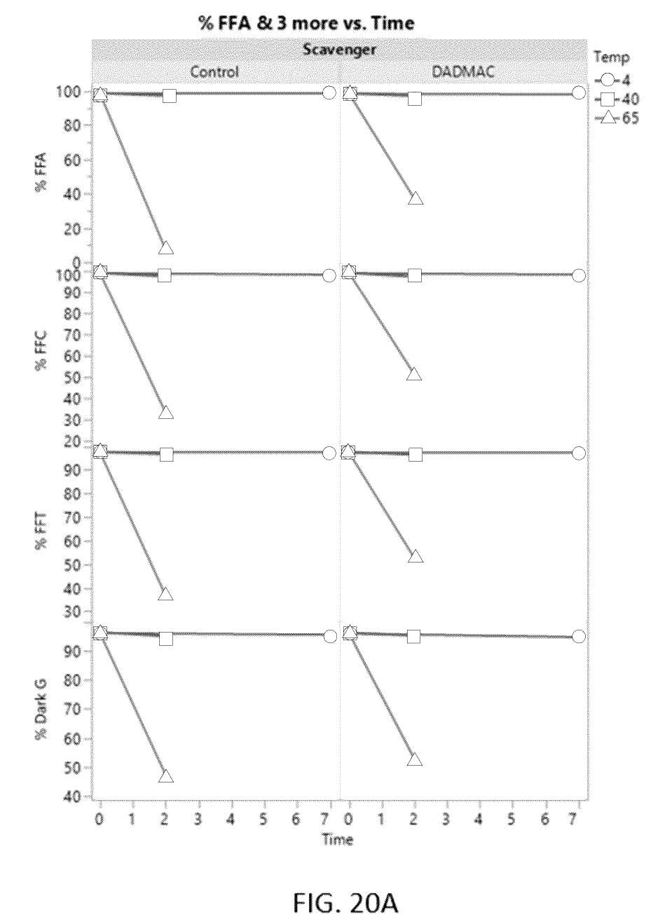

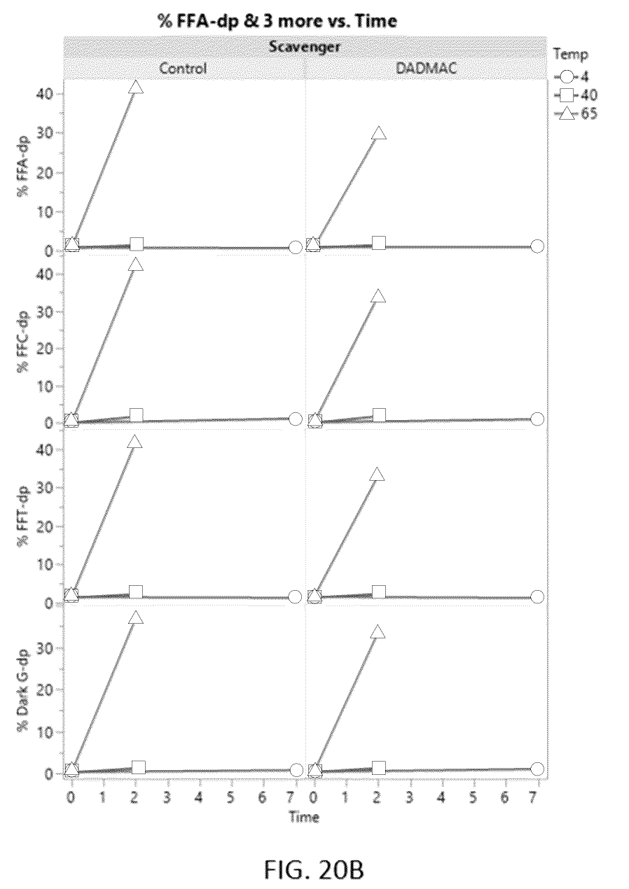

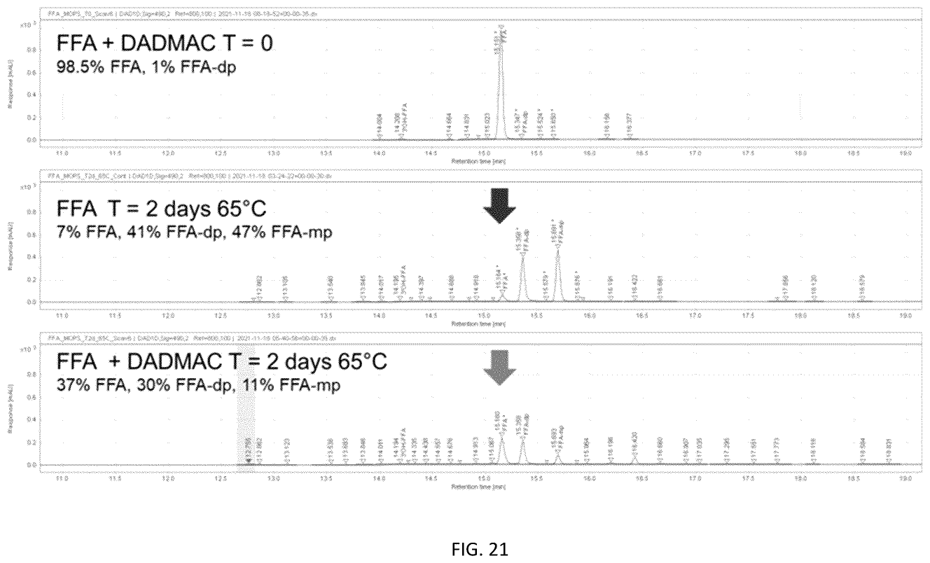

Table 1 shows the ffN purity by HPLC peak area percent and the amount of diphosphate formation (ffN-dp) across each of the ffNs tested, in the absence of DADMAC. Table 2 shows the ffN purity by HPLC peak area percent and the amount of diphosphate formation across each of the ffNs tested, in the presence of DADMAC. FIGS. 20A and 2B provide a graphical representation of the data shown in Tables 1 and 2. FIG. 21 shows that under heat stress an FFA in the presence of DADMAC increased the purity and reduced the percentage of diphosphate and monophosphate production, relative to the control.

TABLE 1
Results of Control (no DADMAC)

| | | FFA- | | FFC- | | FFT- | Dark | Dark |
|---|---|---|---|---|---|---|---|---|
| Condition | FFA | dp | FFC | dp | FFT | dp | G | G-dp |
| 0 days Glycine buffer | 98.75 | 0.78 | 99.11 | nd | 97.85 | 1.43 | 96.48 | 0.33 |
| 0 days MOPS buffer | 98.59 | 0.89 | 99.06 | nd | 97.67 | 1.6 | 96.27 | 0.43 |
| 2 days 40° C. glycine | 97.22 | 1.45 | 97.53 | 1.63 | 96.73 | 2.32 | 95.16 | 1.37 |
| buffer | | | | | | | | |
| 7 days 4° C. MOPS | 98.66 | 0.73 | 98.34 | 1.1 | 97.57 | 1.41 | 95.65 | 0.89 |
| buffer | | | | | | | | |
| 2 days 65° C. MOPS | 7.17 | 41.07 | 33.1 | 42.49 | 36.66 | 41.76 | 47.18 | 36.8 |
| buffer | | | | | | | | |

TABLE 2
Results using 10 mM DADMAC

| | | FFA- | | FFC- | | FFT- | Dark | Dark G- |
|---|---|---|---|---|---|---|---|---|
| Condition | FFA | dp | FFC | dp | FFT | dp | G | dp |
| 0 days Glycine buffer | 98.72 | 0.82 | 99.12 | nd | 97.84 | 1.43 | 96.37 | 0.43 |
| 0 days MOPS buffer | 98.46 | 0.97 | 99.05 | 0.1 | 97.42 | 1.67 | 96.08 | 0.5 |
| 2 days 40° C. glycine | 97.1 | 1.48 | 97.4 | 1.74 | 96.5 | 2.4 | 95.33 | 1.39 |
| buffer | | | | | | | | |
| 7 days 4° C. MOPS | 98.02 | 1.01 | 98.38 | 0.94 | 97.51 | 1.36 | 94.87 | 1.18 |
| buffer | | | | | | | | |
| 2 days 65° C. MOPS | 37.04 | 29.62 | 51.14 | 34.04 | 52.62 | 33.12 | 52.63 | 33.55 |
| buffer | | | | | | | | |

### Example 3—an Example Workflow for Stabilizing ffNs in Sequencing Reactions Using Micelles and Bulky Cation Compounds

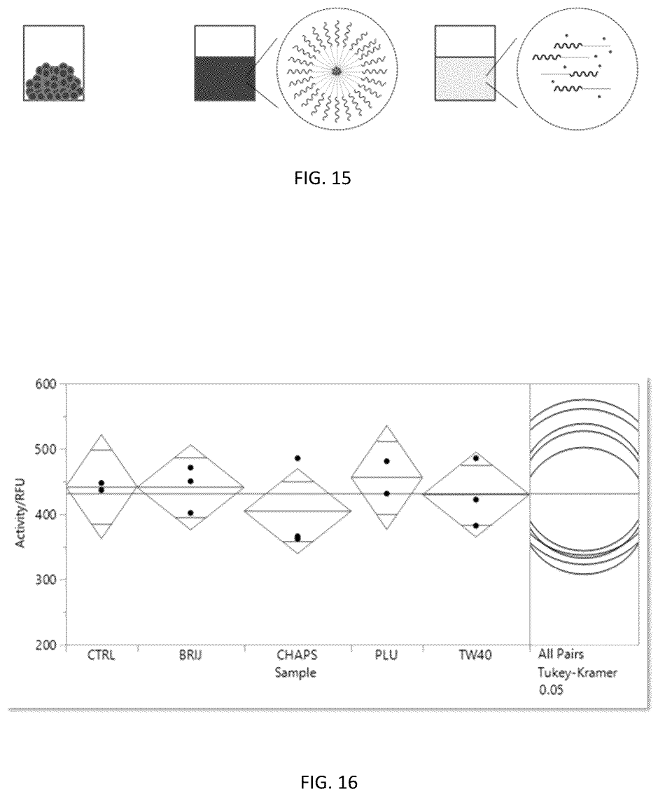

ffNs can be spray-freeze-dried into microspheres or lyophilized cakes. The active ingredients (i.e. ffNs) can be formulated at significantly higher concentrations (e.g. ≥10× [ffN] of full incorporation mix). Resuspension of the microspheres or lyophilized cakes into solution, for on-instrument storage, may then be performed back to/close to pre-lyophilized concentrations. Prior to sequencing, the concentrated ffN solution may then be mixed in with an incorporation mix buffer solution which lowers the concentration of the ffNs, while also diluting the surfactants or the bulky cation compounds. Example workflows are shown in FIG. 15 and in Tables 3 and 4. In Table 3, ffNs are initially lyophilized into microspheres. The lyophilized microspheres are resuspended at a high concentration in solution. In Table 4, the ffNs are diluted in a sequencing mix (IMX) for a sequencing reaction. ffNs are initially lyophilized into microspheres. The lyophilized microspheres are resuspended at a high concentration in solution on-instrument. The ffNs are then diluted in a sequencing mix (IMX) to carry out a sequencing reaction.

TABLE 3
Potential workflows of stabilizing ffNs using surfactants (micelles).

| | Spray-freeze-dried | | |
|---|---|---|---|
| | Microspheres or | Resuspended | Full Incorporation |
| Reagent | Lyophilized Cakes | ffNs in solution | into Sequencing Mix |
| ffN / μM | 10-15,000 | 10-15,000 | 5-9 |
| Surfactant | 0.05-3 | 0.05-3 | 0.015-0.02 |
| (micelle) / % | | | |
| Conc. Factor v. Final | 10x-3,000x | 10x-3,000x | 1x |
| Stage | During storage | On-instrument | During sequencing |

TABLE 4
Potential workflows of stabilizing ffNs using bulky cation compounds.

| | Spray-freeze-dried | | |
|---|---|---|---|
| | Microspheres or | Resuspended | Full Incorporation |
| Reagent | Lyophilized Cakes | ffNs in solution | into Sequencing Mix |
| ffN / μM | 10-15,000 | 10-15,000 | 5-9 |
| Bulky Cation | 1-40 | 1-40 | 0.025-0.4 |
| Compound / mM | | | |
| Conc. Factor v. Final | 10x-200x | 10x-200x | 1x |
| Stage | During storage | On-instrument | During sequencing |

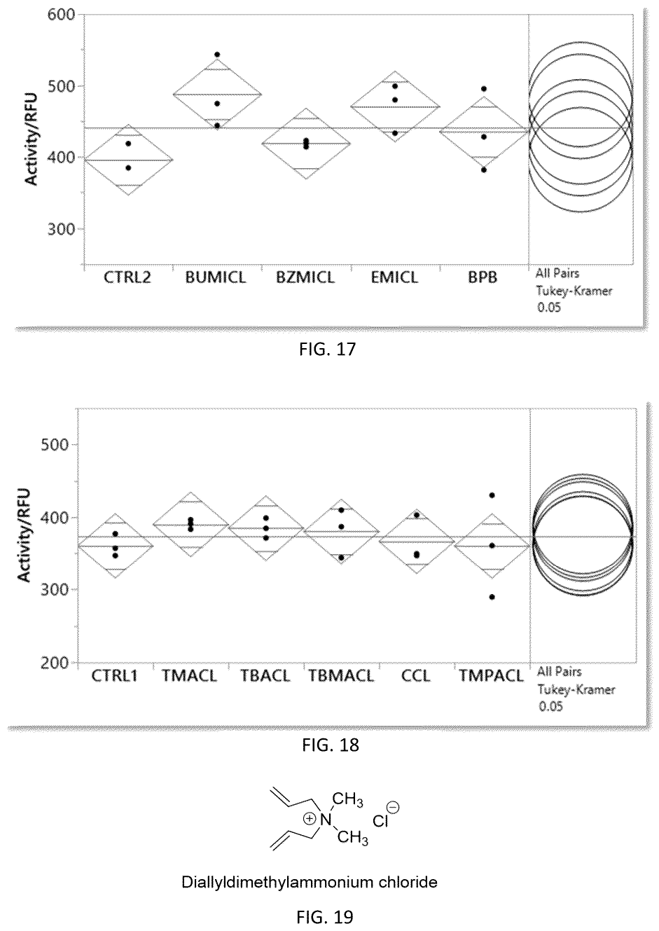

As illustrated by FIGS. 16-18, it was determined that, even when considering the most conservative scenario that is described in Table 2 whereby only a 10× dilution from the microspheres into sequencing mix occurs (i.e. 0.2% surfactant or 3 mM of cation in final solution), incorporation rates of a negative control (CTRL, no surfactant or cation additive) and that of samples containing a bulky cation or surfactant were still statistically equivalent based on the -FRET assay and a Tukey-Kramer analysis.

In FIG. 16:
Brij=tricosaethylene glycol dodecyl ether
PLU=poloxamer 407
TW40=polyoxyethylene sorbitan monopalmitate

### Example 4—Bulky Cation Compounds and Micelles Provide even Greater Protection of Triphosphate as Heat Stress Increases

TBMACL was tested to determine if it provided protection to the triphosphate of ffNs as heat stress increased. ffNs were stored with TBMACL for 35 days. In one of the storage conditions, the temperature was 35° C. and in the other storage condition, the temperature was 45° C.

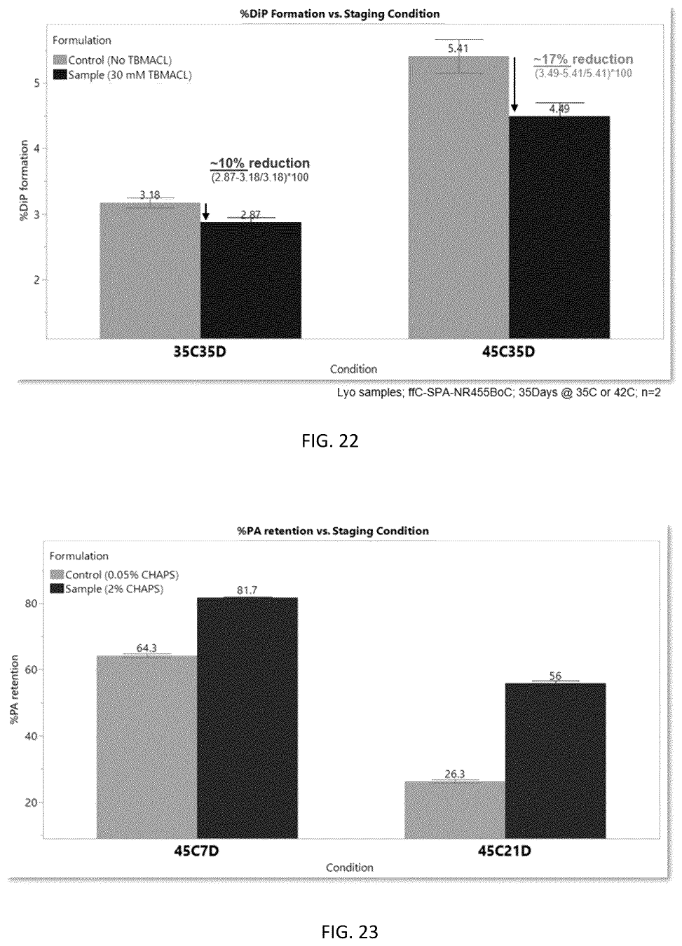

As shown in FIG. 22, there was a greater reduction in dephosphorylation in the presence of TBMACL as the level of heat stress increased. At 35° C. for 35 days TBMACL caused a 10% reduction in diphosphate production. This can be compared to 45° C. for 35 days where TBMACL caused a 17% reduction in diphosphate production. This data suggests that as storage conditions become harsher (i.e., the temperature is increased) bulky cation compounds are more beneficial to triphosphate stability.

CHAPS was tested to determine if it prevented degradation of ffNs as the level of heat stress increased. In one of the reaction conditions ffNs were stored with CHAPS for 7 days at 45° C., and the other reaction condition ffNs were stored with CHAPs for 21 days at 45° C.

As shown in FIG. 23, there was a 27% improvement in percent peak area retention at 7 days, and a 113% improvement in percent PA retention at 21 days. This data shows that as the storage conditions become harsher, surfactants are more beneficial to maintaining intact ffNs.

### Example 5—Workflows for Stabilizing ffNS During Storage

Bulky cation compounds and micelles described herein can be used to store ffNs.

Long Term Storage: A reagent can be manufactured in dry state with stabilizing additives to enable long term storage. The dried reagent can include bulky cation compounds or surfactants.

On-Instrument Storage: A dried reagent can be resuspended in liquid in the instrument and stored for the length of multiple runs. A “multi-run” equals one resuspended, concentrated, compact bulk reagent and can be used to feed multiple sequencing runs. Resuspension of lyophiles adds time to the sequencing run. Workflows described in Example 3 enable one resuspension event per multiple sequencing runs to reduce run times.

In-Use for Chemistry: Concentrated liquid is diluted on-board to optimize concentration for activity. Onboard dilution takes less than resuspension of dry lyophiles to a liquid state.

Table 5 provides storage conditions and storage durations of each for each of these storage conditions

TABLE 5
Storage conditions for long term storage, on-instrument storage, and in-use chemistry

| | Long Term Storage | On-Instrument Storage | In-Use for Chemistry |
|---|---|---|---|
| Length of estimated | ~years | ~months | ~days |
| stability | (potentially 1-2 years) | (potentially 1-3 months) | (potentially 1-2 days) |
| Format | Concentrated dry lyophiles | Resuspend lyophiles to | Diluted liquid |
| | | concentrated liquid | |
| Example | 100X | 50-100x | 1x |
| concentration | (30 mM cation) | (15-30 mM cation and/or | (0.3-3 mM cation and/or |
| factor | | 1-2% detergent) | 0.02%-0.2% detergent) |

| | | |
|---|---|---|
| Description | Additives at sufficiently high | Additive diluted |
| | concentration to have stabilizing effect | sufficiently low to |

prevent interference
chemistry

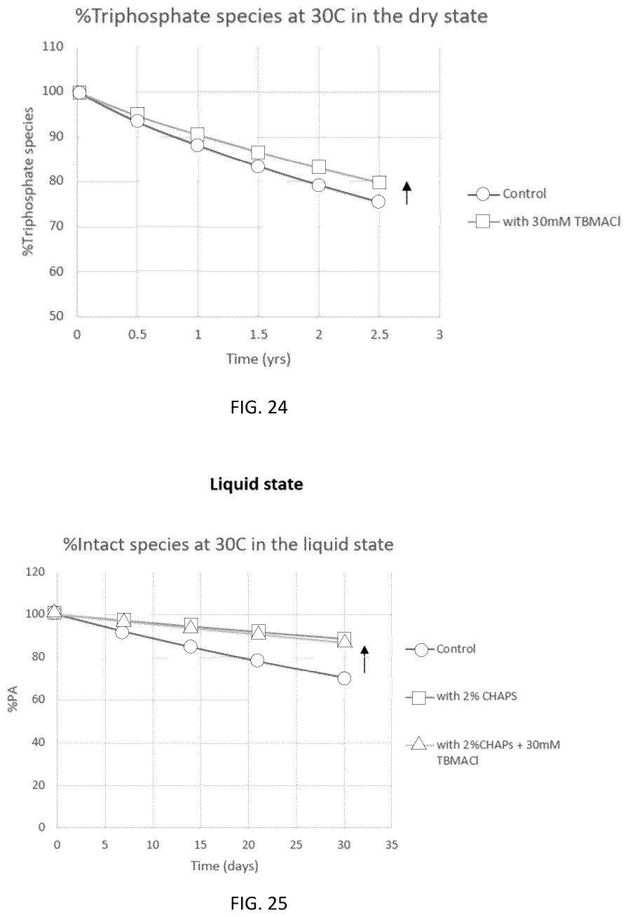

FIG. 24 shows that adding the bulky cation compound TBMACL to ffNs in a dry state increased stability of the trisphosphate species. Also, FIG. 25 shows that addition of the surfactant CHAPs to ffNs in a liquid state, increased the stability of trisphosphate species.

### Example 5—Inhibiting Bacterial Growth

TBMACL was added to incorporation mix as an ffN stabilizer, and exhibited biocidal properties. The data included here demonstrate that TBMACL has a dose-dependent biocidal effect on bacterial growth, and it consequently has biocidal properties in liquid reagents.

Therefore, TBMACL may be used both as a sequencing-compatible ffN stabilizer and as a biocide to reduce or eliminate the need for inclusion of another (biocidal) compound.

To confirm the biocidal effect of TBMACl within liquid sequencing reagents, a titration of TBMACL from 9 mM to 90 mM in incorporation mix was prepared. These experiments were carried out with the omission of polymerase and ffNs in the formulation, as they were not required for the further analysis. On day 0, samples were inoculated with 3×10⁶ CFU/ml of the bacteria Arthrobacter russicus. The samples were staged at 12° C. for 0, 14 and 30 days, followed by plating to determine CFU/ml.

FIGS. 26-27 provide data showing use of a bulky cationic compound to inhibit bacterial growth. More specifically, FIG. 26 illustrates A. russicus growth in a titration of TBMACL over 30 days at 12° C.; the data shown is on a linear Y axis. FIG. 27 illustrates the same data, with a logarithmic scale Y axis. It may be understood from FIGS. 26-27 that the greater the concentration of TBMACL, the greater the inhibition of bacterial growth. For example, the sample with 0 mM TBMACL first showed a small decrease in bacterial growth at day 14, followed by a sharp increase in bacterial growth at day 30. In the absence of TBMACL, the CFU/ml increase by about 1.8-fold over 30 days. The sample with 9 mM TBMACL first showed a small increase in bacterial growth at day 14, followed by a decrease in bacterial levels back to approximately the starting level—an approximately static effect on the bioburden load. The samples with 22.5 mM, and 45 mM TBMACL showed steep declines in bacterial growth at day 14, followed by more modest declines at day 30. These concentrations had a moderate bactericidal effect, reducing CFU/ml by 6 and 8.6-fold respectively. The sample with 90 mM TBMACL appeared to kill essentially all bacteria by day 14, and all bacteria appeared to remain dead at day 30. This concentration of TBMACL had a strong bactericidal effect, reducing CFU/ml by about 78,000-fold.

The data shown in FIGS. 26-27 confirm that the addition to TBMACL to liquid incorporation mix has a dose-dependent effect on reducing CFU/ml of A. russicus and therefore the bioburden load. From these data, it is expected that a concentration of at least about 90 mM (e.g., about 90 mM to about 1000 mM) of TBMACL to liquid incorporation mix may inhibit bacterial growth, in addition to other benefits such as described elsewhere herein.

### Additional Comments

While various illustrative examples are described above, it will be apparent to one skilled in the art that various changes and modifications may be made therein without departing from the disclosure. The appended claims are intended to cover all such changes and modifications that fall within the true spirit and scope of the examples provided herein.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method of stabilizing fully functional nucleotides (ffNs), the method comprising:
including the ffNs and at least one surfactant into lyophilized material.

2. The method of claim 1, wherein a concentration of the ffNs is between about 10 μM and about 15,000 μM.

3. The method of claim 1, wherein a concentration of the at least one surfactant is between about 0.5% and about 2.5%.

4. The method of claim 1, wherein including the ffNs and the at least one surfactant into the lyophilized material comprises freeze-drying the ffNs.

5. The method of claim 1, wherein the ffNs are encapsulated in a hydrophobic core of the surfactant, which inhibits or prevents hydrolysis of the ffNs.

6. The method of claim 1, wherein the at least one surfactant comprises any one or more of tricosaethylene glycol dodecyl ether, poloxamer 407, polyethylene glycol (PEG) stearate, SB-12, lauramidopropyl betaine, n-dodecyl-β-D-maltoside, CHAPS, polyoxyethylene sorbitan monolaurate, and polyoxyethylene sorbitan monopalmitate.

7. A method of stabilizing a fully functional nucleotides (ffNs), the method comprising:
including the ffNs and at least one bulky cation compound into lyophilized material.

8-17. (canceled)

18. A method of preparing fully functional nucleotides (ffNs) for sequencing, the method comprising:
mixing the ffNs with at least one surfactant in a solution;
diluting a concentration of the ffNs in a sequencing mix relative to a concentration of the ffNs in the solution by a factor of between about 10× and about 200×; and
diluting a concentration of the at least one surfactant in the sequencing mix, relative to a concentration of the at least one surfactant in the solution, by a factor of between about 10× and about 1,000×.

19. The method of claim 18, wherein the concentration of the ffNs in the solution is between about 10 μM and about 15,000 μM, and the concentration of the ffNs in the sequencing mix is between about 5 μM and about 9 μM.

20. The method of claim 18, wherein the concentration of the at least one surfactant in the solution is between about 1.5% and about 2.5%, and the concentration of the at least one surfactant in the sequencing mix is between about 0.05% and about 0.25%.

21. The method of claim 18, wherein the at least one surfactant comprises any one or more of tricosaethylene glycol dodecyl ether, poloxamer 407, CHAPS, polyoxyethylene sorbitan monolaurate, and polyoxyethylene sorbitan monopalmitate.

22. The method of claim 18, further comprising performing a sequencing reaction through incorporating the ffNs onto a nucleic acid.

23. The method of claim 22, wherein the sequencing reaction comprises a sequencing by synthesis reaction.

24. A method of preparing ffNs for sequencing, the method comprising:
mixing the ffNs with at least one bulky cation compound in a solution;
diluting a concentration of the ffNs in a sequencing mix relative to a concentration of the ffNs in the solution by a factor of between about 10× and about 200×; and
diluting the concentration the at least one bulky cation compound in the sequencing mix relative to the concentration of the at least one bulky cation compound in the solution, by a factor of between about 100× and about 1,000×.

25-29. (canceled)
